(19) United States
(12) Patent Application Publication
(10) Pub. No.: US 2004/0229913 A1
(43) Pub. Date: Nov. 18, 2004

(54) Therapeutic agent for severe Alzheimer's dementia

(75) Inventors: Birol Emir, New York, NY (US); Ponni Subbiah, New York, NY (US)

Correspondence Address: WILMER CUTLER PICKERING HALE AND DORR LLP, THE WILLARD OFFICE BUILDING, 1455 PENNSYLVANIA AVE, NW, WASHINGTON, DC, 20004, US

(73) Assignee: Eisal Co., Ltd., Tokyo (JP)

(21) Appl. No.: 10/622,769

(22) Filed: Jun. 28, 2004

(30) Foreign Application Priority Data
Dec. 13, 2002 (JP) 2002-363139

(51) Int. Cl. A61K 31/445

(52) U.S. Cl. 514/319

(57) ABSTRACT

The object is to offer an effective therapeutic agent for severe Alzheimer's dementia. The above problem is solved with a therapeutic agent comprising as an active ingredient 1-benzyl-4-[(5,6-dimethoxy-1-indanon)-2-yl]methylpiperidine or a pharmaceutically acceptable salt thereof.

### Related Applications

This application claims priority to Japanese Patent Application No. 2002-363139 filed Dec. 13, 2002.

### FIELD OF THE INVENTION

The invention relates to a therapeutic agent for Alzheimer's dementia and more specifically relates to a therapeutic agent for severe Alzheimer's dementia with donepezil as an active ingredient.

### BACKGROUND OF THE INVENTION

It is reported that four to five percent of elderly persons 65 years or older in Japan (approximately 1 million people) have dementia and that this is at least 20% in persons 85 years or older (\overline{O}tsuka T., Senile Dementia 8 (3): 283-290, 1994). It is expected that the number of elderly persons with dementia will increase together with the expanding elderly population. The growing elderly population, not only in Japan but worldwide as well, is therefore a dire medical and social problem.

Alzheimer's dementia is known as a form of dementia characteristic of dementia illnesses that appear in old age. Alzheimer's dementia is pathologically characterized by a generalized atrophy of the brain, the appearance of senile plaques of the cerebral cortex, and changes to the Alzheimer's neurofibrils. Also, neurochemistry research efforts have discovered that in this form of dementia, there are abnormalities in cholinergic neurons, and it is reported that there is a decrease in acetylcholine and a drop in the activity of choline acetyltransferase, an acetylcholine synthetase mainly in the forebrain (Perry P. K. et al., Lancet 1 189, 1977).

While psychotropic and other drugs are somewhat effective on the peripheral symptoms of Alzheimer's dementia, there are no known drugs that are effective against the central symptoms of dementia, such as the loss of memory, cognition, and decision-making ability.

But it has recently been disclosed that donepezil, shown in Formula (I) below, or a pharmaceutically acceptable salt thereof, as a therapeutic agent for mild and moderate Alzheimer's senile dementia, is useful as a therapeutic and preventative agent for senile dementia (Japanese unexamined patent application publication H1-79151), and donepezil hydrochloride is commercially available as an actual drug (product name: ARICEPT® by Eisai Co., Ltd.).

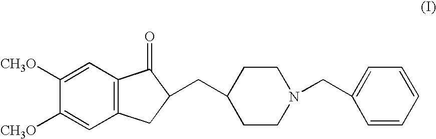

With the trend toward an elderly society already mentioned, demand is markedly growing for a therapeutic agent that goes beyond treating mild and moderate Alzheimer's dementia to also be effective on severe Alzheimer's dementia characterized by advanced dementia.

One difference between severe Alzheimer's dementia and mild/moderate Alzheimer's dementia is that the former involves a loss of neural structure within the brain, a marked drop in brain volume, and a significant decrease in blood flow within the brain. It can therefore be said the pathology associated with severe Alzheimer's dementia is completely different from those of mild/moderate Alzheimer's dementia.

There are, however, no promising therapeutic agents for severe Alzheimer's dementia.

One obstacle hindering the development of therapeutic agents effective against severe Alzheimer's dementia is the lack of a reliable method to evaluate improvement in dementia. A known conventional method for evaluating improvement in dementia for mild and moderate Alzheimer's dementia is ADAS-cog, which was developed to evaluate cognitive impairment in Alzheimer's dementia.

ADAS-cog, an abbreviation for Alzheimer's Disease Assessment Scale, cognitive subscale, is an 11-item scale for evaluating cognitive dysfunction in the areas of memory, orientation, language, and understanding. The higher the score, which ranges from 0 to 70 points, the more severe the cognitive impairment.

The 11 items are word recall, spoken language ability, understanding of speech, word-finding difficulty, following oral commands, naming of fingers and objects, constructional praxis, ideational praxis, orientation, word recognition, and recall of test instructions.

ADAS-cog is also employed as a method to evaluate cognitive function in clinical assessment guidelines in Europe and the United States, and is most widely used as a standardized test of cognitive function.

But as the level of dementia in severe Alzheimer's dementia is great and, as was mentioned, involves pathology very different from that of mild and moderate Alzheimer's dementia, it is not possible to evaluate improvement in severe Alzheimer's dementia with ADAS-cog. Therefore, no therapeutic agents effective against severe Alzheimer's dementia have been developed.

### SUMMARY OF THE INVENTION

An objective of the inventors, in light of the above circumstances, was thus to provide a therapeutic agent effective against severe Alzheimer's dementia based on an evaluation method completely different from that associated with mild and moderate Alzheimer's dementia.

The object was achieved with a therapeutic agent for severe Alzheimer's dementia comprising as an active ingredient the compound 1-benzyl-4-[(5,6-dimethoxy-1-indanon)-2-yl]methylpiperidine of Formula (I) below or a pharmaceutically acceptable salt thereof.

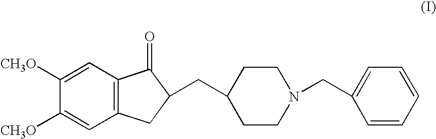

Additionally in a preferred embodiment of the invention, the therapeutic agent is characterized as comprising 3.0 to 10.0 mg of the above compound per dosage unit.

The term “therapeutic agent” used in the context of the invention, refers not only to an agent for suppressing the progress of symptoms, but also to an agent for improving symptoms.

### BRIEF DESCRIPTION OF THE DRAWING

FIG. 1 shows the results of a method of SIB evaluation used in the invention of patients with severe Alzheimer's dementia (MMSE<10). In the analysis with the evaluation method, a significant difference was observed at p<0.05 with Fisher's least significant difference test, two sided.

### DETAILED DESCRIPTION OF THE INVENTION

Hereafter, the invention will be described in detail, but the technical scope of the invention is not limited to the descriptions herein.

The compound used in the invention—1-benzyl-4-[(5,6-dimethoxy-1-indanon)-2-yl]methylpiperidine, expressed by Formula (I) below, or a pharmaceutically acceptable salt thereof—was discovered to be useful in the treatment of severe Alzheimer's dementia.

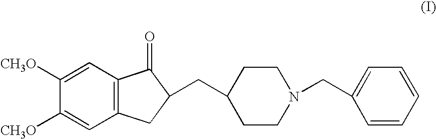

With regard to methods for producing the compound used in the invention, this compound can be produced using the production method discussed in Japanese unexamined patent application publication H1-79151 or another known method, and can be easily produced by appropriately selecting the reactants, reagents, and reaction conditions.

Examples of pharmaceutically acceptable salts in the invention are salts of an inorganic acid, salts of an organic acid, salts of an inorganic base, salts of an organic base, and salts of an acidic or basic amino acid. An acid or base is formed into a salt at an appropriate ratio of 0.1 to 5 molecules per 1 molecule of the compound.

Preferable examples of salts of inorganic acids are salts of hydrochloric acid, hydrobromic acid, sulfuric acid, nitric acid, and phosphoric acid; preferable examples of salts of organic acids are salts of acetic acid, succinic acid, fumaric acid, maleic acid, tartaric acid, citric acid, lactic acid, stearic acid, benzoic acid, methansulfonic acid, and p-toluene sulfonic acid.

Preferable examples of salts of inorganic bases are a salt of sodium, alkaline metals such as potassium, a salt of calcium, alkaline earth metals such as magnesium, a salt of aluminum, and an ammonium salt. Preferable examples of salts of organic bases are salts of diethylamine, diethanolamine, meglumine, and N, N′-dibenzyl ethylene diamine.

Preferable examples of salts of acidic amino acids are salts of aspartic acid and glutamic acid; preferable examples of salts of basic amino acids are salts of arginine, lysine, and omithine. In the invention, donepezil hydrochloride, a hydrochloride salt expressed with the Formula (II) below, is particularly useful in the treatment of severe Alzheimer's dementia.

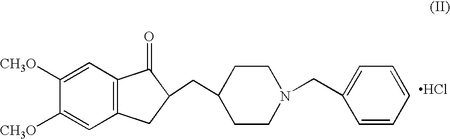

Note the above compound, which is used in the invention, has an asymmetric carbon and can thus exist as an optically active form, but the compound used in the invention can be any optically active form, a mix of stereoisomers such as diastereoisomers, or a racemic mixture.

The dosage route of the compound used in the invention when used as a drug is not particularly limited and may be oral or non-oral (e.g., intramuscularly administered, intravenously administered, subcutaneously administered, intraperitoneally administered, percutaneously administered, administered to the mucous membranes of the sinuses or another location, or inhaled).

The dose of the drug contained in the compound used in the invention can be appropriately selected upon the overall consideration of the severity of the symptoms, the age, sex, and weight of the patient, differences in sensitivity, the method of administration, the time of administration, the interval between doses, and the properties and preparation of the pharmaceutical formulation. There are no particular limits to the dose, but the daily dose for a normal adult is about 0.1 to 100 mg, preferably about 0.3 to 50 mg, and more preferably from 0.5 to 10 mg. It is, in normal cases, administered in 1 to 4 allotments daily. A total dose when given in one daily allotment of 0.1 mg or less brings no therapeutic effect, and a total dose when given in four daily allotments of 100 mg or greater results in an elevated blood concentration of the drug and the danger of vomiting and other symptoms.

Note that donepezil-based compounds, which are used in the invention, brought about no serious toxicity at doses of about 100 mg/kg and above when given to rats in toxicity studies.

There are no particular limits to the dosage form of the drug of the invention, and formulations for oral dosage include tablets, capsules, fine granules, powder, granules, orally disintegrating tablets, liquids, and syrups; formulations for non-oral administration include injectable formulations, formulations for intravenous drip, suppositories, inhalants, transdermally absorbed formulations, transmucosally absorbed formulations, nasal drops, and ear drops. Tablets are desirable as an oral dosage form for patients with dementia.

The usefulness of the compound expressed by formula (II) in severe Alzheimer's dementia is explained with the following test methods.

First, a method for distinguishing patients suffering from severe Alzheimer's dementia from patients with dementia will be discussed. With regard to the standards for distinguishing such patients, MMSE, objective standards for gauging the severity of impairment to cognitive function, was used as a scale of the severity of Alzheimer's dementia. MMSE is the acronym for Mini-Mental State Examination and is a simple cognitive function testing method that includes some behavioral aspects in addition to memory. MMSE is in wide use internationally and tests using simple questions about time, location, and the naming of objects. In the test, the severity of dementia is indicated with a score from 30 to 0—the closer the score to 30, the more normal the cognitive function, while the closer the score to 0, the greater the severity of impairment to cognitive function.

In the invention, patients with a score of 5 to 9 in the MMSE test were considered to be patients with severe Alzheimer's dementia. Note that the MMSE scores of patients with moderate Alzheimer's dementia range from 10 to 17.

Next, a method for evaluating improvement in severe Alzheimer's dementia used in the invention is discussed. SIB (abbreviation for severe impairment battery) is used as a method for evaluating severely impaired cognitive function. SIB is composed of the nine items of social interaction: memory, orientation, attention, praxis, visuospatial ability, language, construction, and orientation to name, and evaluation is performed through patient interviews with a total of 100 possible points. Each question is a simple evaluation method that allows assessment of the lowest range of cognitive function corresponding to a severely impaired level. The lower the score determined through SIB, the greater the impairment to cognitive function.

Score is assigned on a three-level scale for the questions asked for each item. In greater detail, a correct answer is worth two points, a partially correct answer (e.g., a mostly correct reply) is worth 1 point, and an incorrect answer is worth zero points. A score of one point indicates whether or not the patient is able to accurately respond through gesture or counseling.

Specifically, in the area of social interaction, a patient who in response to a request to “Please sit here” sits on her own in a chair would earn 2 points as a correct answer, a patient who sits upon urging would earn 1 point, and a patient who did anything else would be charged with an incorrect answer. In the area of orientation, a patient who in response to the request “Please state the days of the week” answers correctly would earn 2 points as a correct answer, and a patient who answers correctly after being counseled up to 2 times or who makes one or two mistakes would only earn 1 point. Any other response earns zero points. And in the area of construction, a patient who, in response to the request “Please draw a circle” draws a circle, an egg-shaped form, or an oval on his own, would earn 2 points as a correct answer, and a patient who draws a similar shape, for example, at least a semicircle, or who draws a circle upon counseling would earn 1 point. Any other response earns zero points.

An evaluation of cognitive function through SIB is performed as the total score. Any increase in score constitutes an index of improvement in cognitive function, while a decrease in score indicates a drop in cognitive function.

Also, CIBIC plus (the abbreviation for Clinician's Interview-Based Impression of Change plus)—a scale to evaluate overall clinical symptoms used to evaluate improvement in Alzheimer's dementia in the invention—is briefly discussed. In CIBIC plus, a patient's guardian or family is interviewed and then the patient herself is interviewed to obtain a comprehensive assessment.

The evaluation items are, specifically, the four fields of overall impression, cognition, actions, and daily activities; they are assessed on the seven-level scale of substantial improvement, improvement, slight improvement, no change, slight deterioration, deterioration, and substantial deterioration as an overall evaluation through comparison with the baseline state.

A double-blind, comparative study with placebo or donepezil hydrochloride formulation given once daily for 24 weeks was performed with patients determined, through SIB, to have severe Alzheimer's dementia.

In the study, the dosing period was set based on the determination that an assessment of the efficacy of the drug in 24-week placebo-controlled administration would be necessary in consideration of the expectation that dementia medications are continuously administered over the long term. The double-blind comparative study was a comparative study in which neither the physicians nor the patients were informed of whether the donepezil hydrochloride-containing tablets or the placebo tablets were being given.

The invention is described in greater detail through the following working example, but the scope of the invention is in no way limited thereto.

### EXAMPLES

### Example Study 1

SIB Evaluation

Placebo or a formulation comprising donepezil hydrochloride (hereinafter referred to as “E2020”) was administered as stated hereinafter to patients with severe Alzheimer's dementia scoring 5 to 9 on MMSE. In greater detail, tablets not containing E2020 (hereinafter referred to as “placebo tablets”) and tablets comprised of 10 mg of E2020 (hereinafter referred to as “E2020 tablets”) were orally administered for a 24-week period once daily. However, tablets with 5 mg of E2020 were given as the E2020 tablets for the first four weeks.

Table 1 presents the backgrounds of the patients included in the study of the invention. As Table 1 shows, there was little difference in the percentage of the ages or sexes or body weight between the sexes of the patients given the E2020 tablets or the placebo tablets.

ADAS-cog, which has been used to evaluate the symptoms of mild and moderate Alzheimer's dementia, could not be used as a method to evaluate improvement in dementia of patients with severe Alzheimer's dementia. This is because demented patients cannot understand the complex question contents of ADAS-cog.

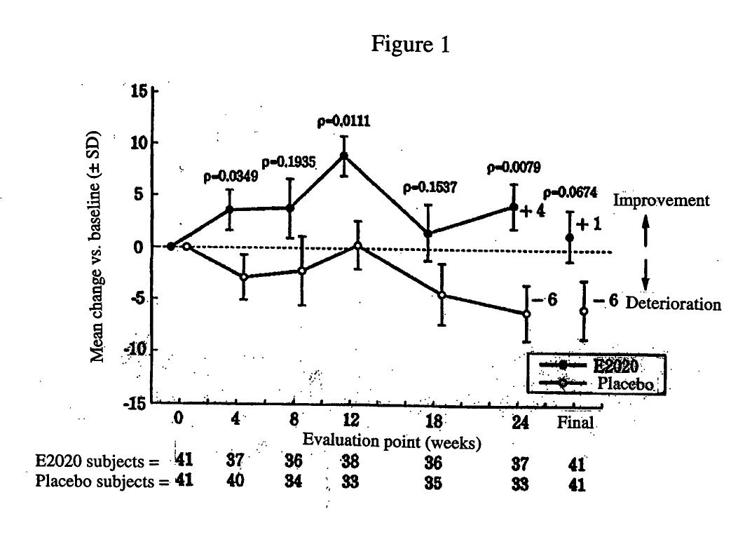

FIG. 1 shows the results of SIB evaluation of severe Alzheimer's dementia patients scoring 5 to 9 on the MMSE who were given the E2020 tablets or the placebo tablets. The horizontal axis in FIG. 1 represents the week of dosing, and the vertical axis in FIG. 1 shows baseline SIB scores and the mean change in SIOB scores following the start of administration. A mean change of zero or greater on the vertical axis of FIG. 1 indicates an improvement in the symptoms of dementia, while a mean change of zero or less indicates a deterioration in the symptoms of dementia.

Note that the term “final” evaluation period means both the 4-week period following discontinuation after administration for a 24-week period as well as the 16- or 10-week period following discontinuation after administration for a 12- or 18-week period.

As FIG. 1 clearly shows, a trend of improvement was noted 4 weeks after the start of E2020 administration, and the maximum improvement in the study was seen 12 weeks after the start of administration.

In greater detail, the mean changes at the 24-week and final evaluation periods in those given the E2020 tablets were +4 points and +1 point, respectively, which indicates a trend of improvement.

On the other hand, those given the placebo tablets showed no trend of improvement during the 24-week period after the start of administration. The mean changes at the 24-week and final evaluation periods in those given the placebo tablets were -6 points each, which indicates a trend of deterioration.

A comparison based on the SIB results of the patients receiving the E2020 tablets to those receiving the placebo tablets revealed a significant difference at the 4-, 12-, and 14-week evaluation points. The administration of the E2020 tablets brought improvement to the symptoms of severe Alzheimer's dementia. Therefore, the E2020 tablets were determined to be a useful therapeutic agent for severe Alzheimer's dementia.

Although it is clear from the results of SIB evaluation that E2020 is a useful compound in the treatment of severe Alzheimer's dementia, the results of the effects of treatment of severe Alzheimer's dementia with E2020 obtained through the following evaluation method are presented.

### Example Study 2

CIBIC Plus Evaluation

Table 2 shows the results of a method of CIBIC plus evaluation used in the invention of patients with severe Alzheimer's dementia (MMSE <10). The Cochran-Mantel-Haenszel test was used in the analysis of the evaluation method.

Table 2 shows the results of CIBIC plus evaluation of severe Alzheimer's dementia patients scoring 5 to 9 on the MMSE who were given the E2020 tablets or the placebo tablets. The assessment items in the table are the results of overall evaluation on the seven-level scale, while the numbers in parentheses are figures representing the percent (%) of the evaluation classification to the total number of subjects evaluated. The evaluation points were, as was the case with SIB evaluation, week 4, week 8, week 12, week 18, week 24, and final evaluation.

The rightmost column of Table 2 contains the total number of assessments of no change, slight improvement, improvement, and substantial improvement (hereafter referred to as “no change or better”) along with the percentage (in parentheses) to the total number of subjects in the treatment group in question. The percentage of no change or better for the E2020 group was considerably higher than that of the placebo group at each evaluation point, and an improvement in the symptoms of severe Alzheimer's dementia was noted in the E2020 group.

There was a significant difference between the E2020 group and the placebo group at the evaluation points of week 4, week 8, and week 18 in particular. In greater detail, 92% of the E2020 group was given a rating of no change or better at the week 4 evaluation point, while this was 60% in the placebo group. At the week 8 evaluation point, 86% of the E2020 group was given a rating of no change or better, while this was 47% in the placebo group. And at the week 18 evaluation point, 73% of the E2020 group was given a rating of no change or better, while this was 38% in the placebo group.

A look at the E2020 group reveals that the percentage of the group given a rating of no change or better at the week 4, 8, 12, 18, and 24 evaluation points was, respectively, 92%, 86%, 85%, 73%, and 49%. And the percentage of the placebo group given a rating of no change or better at the week 4, 8, 12, 18, and 24 evaluation points was, respectively, 60%, 47%, 52%, 38%, and 36%—a trend of decline was noted throughout the study.

The slight variation in the numbers of subjects at each evaluation point in FIG. 1 and Table 2 is due to the presence of subjects who were not included in evaluation in one study or the other.

An analysis of the above confirms an improvement in severe Alzheimer's dementia in the E2020 group.

It was confirmed from the results of the studies that donepezil hydrochloride, a compound used in the invention, is effective in the treatment of severe Alzheimer's dementia.

According to the invention—with the determination that donepezil hydrochloride is a useful therapeutic agent for severe Alzheimer's dementia—a therapeutic agent for severe Alzheimer's dementia with donepezil hydrochloride as an active ingredient can be offered.

TABLE 1
Patient Backgrounds

| Administration category | E2020 | Placebo |
|---|---|---|
| Age | 73.3 ± 8.5 | 74.0 ± 7.8 |
| Mean ± SD (n) | | |
| Sex | Male 56 (38.9%) | Male 57 (39.0%) |
| | Female 88 (61.1%) | Female 89 (61.0%) |
| Body weight | Male 72.9 ± 11.2 | Male 75.6 ± 10.4 |
| Mean ± SD (n) | Female 59.5 ± 11.8 | Female 58.8 ± 11.1 |

TABLE 2

| | | | | | | | | | | | | No |
|---|---|---|---|---|---|---|---|---|---|---|---|---|
| | | | | | | | | | | | | change |
| Eval. | Eval. std. | | Subst. | | Slight | No | Slight | | Subst. | | Intra-grp. | or |
| Point | Dosage grp. | | imp. | Imp. | imp. | change | deter. | Deter. | deter. | Total | compar.* | better |
| Week | E2020 | # sub. | 0 | 0 | 14 | 22 | 3 | 0 | 0 | 39 | 0.0012* | 36 |
| 4 | | (%) | (0) | (0) | (36) | (56) | (8) | (0) | (0) | | | (92) |
| | Placebo | # sub. | 0 | 0 | 6 | 18 | 13 | 2 | 1 | 40 | | 24 |
| | | (%) | (0) | (0) | (15) | (45) | (33) | (5) | (3) | | | (60) |
| Week | E2020 | # sub. | 0 | 1 | 16 | 14 | 4 | 1 | 0 | 36 | 0.0202* | 31 |
| 8 | | (%) | (0) | (3) | (44) | (38) | (11) | (3) | (0) | | | (86) |
| | Placebo | # sub. | 0 | 1 | 7 | 8 | 16 | 1 | 1 | 34 | | 16 |
| | | (%) | (0) | (3) | (21) | (24) | (47) | (3) | (3) | | | (47) |
| Week | E2020 | # sub. | 0 | 3 | 14 | 16 | 5 | 1 | 0 | 39 | 0.0753 | 33 |
| 12 | | (%) | (0) | (8) | (38) | (41) | (13) | (3) | (0) | | | (85) |
| | Placebo | # sub. | 0 | 1 | 8 | 8 | 13 | 3 | 0 | 33 | | 17 |
| | | (%) | (0) | (3) | (24) | (24) | (39) | (9) | (0) | | | (52) |
| Week | E2020 | # sub. | 0 | 0 | 13 | 14 | 9 | 1 | 0 | 37 | 0.0101* | 27 |
| 18 | | (%) | (0) | (0) | (35) | (38) | (24) | (3) | (0) | | | (73) |
| | Placebo | # sub. | 0 | 0 | 6 | 7 | 16 | 5 | 0 | 34 | | 13 |
| | | (%) | (0) | (0) | (18) | (21) | (47) | (15) | (0) | | | (38) |
| Week | E2020 | # sub. | 0 | 1 | 8 | 9 | 17 | 2 | 0 | 37 | 0.2867 | 18 |
| 24 | | (%) | (0) | (3) | (22) | (24) | (46) | (5) | (0) | | | (49) |
| | Placebo | # sub. | 0 | 0 | 6 | 6 | 14 | 7 | 0 | 33 | | 12 |
| | | (%) | (0) | (0) | (18) | (18) | (42) | (21) | (0) | | | (36) |
| Final | E2020 | # sub. | 0 | 1 | 9 | 9 | 20 | 3 | 0 | 42 | 0.1780 | 19 |
| | | (%) | (0) | (2) | (21) | (21) | (48) | (7) | (0) | | | (45) |
| | Placebo | # sub. | 0 | 0 | 6 | 6 | 18 | 10 | 1 | 41 | | 12 |
| | | (%) | (0) | (0) | (15) | (15) | (44) | (24) | (2) | | | (29) |

(*: p < 0.05)

1-2. (Canceled)

3. A method for treating severe Alzheimer's dementia in a patient in need thereof comprising administering 3.0 mg to 10 mg of 1-benzyl-4-(5,6-dimethoxy-1-indanon)2-yl)methylpiperidine or a pharmaceutically acceptable salt thereof to treat severe Alzheimer's dementia in the patient; wherein severe Alzheimer's dementia is characterized by a score of 5 to 9 on the Mini-Mental State Examination.

4. A method for treating severe Alzheimer's dementia in a patient in need thereof comprising administering 10 mg of 1-benzyl-4-(5,6-dimethoxy-1-indanon)2-yl)methylpiperidine hydrochloride to treat severe Alzheimer's dementia in the patient; wherein severe Alzheimer's dementia is characterized by a score of 5 to 9 on the Mini-Mental State Examination.

5. A method for treating severe Alzheimer's dementia in a patient in need thereof comprising administering a therapeutically effective amount of 1-benzyl-4-(5,6-dimethoxy-1-indanon)2-yl)methylpiperidine or a pharmaceutically acceptable salt thereof to treat severe Alzheimer's dementia in the patient.

6. The method of claim 5, comprising administering 0.3 mg to 50 mg of 1-benzyl-4-(5,6-dimethoxy-1-indanon)2-yl)methylpiperidine or a pharmaceutically acceptable salt thereof to treat severe Alzheimer's dementia in the patient.

7. The method of claim 6, comprising administering 3.0 mg to 10.0 mg of 1-benzyl-4-(5,6-dimethoxy-1-indanon)2-yl)methylpiperidine or a pharmaceutically acceptable salt thereof to treat severe Alzheimer's dementia in the patient.

8. The method of claim 5, wherein severe Alzheimer's dementia is characterized by a score of less than 10 on the Mini-Mental State Examination.

9. The method of claim 8, wherein severe Alzheimer's dementia is characterized by a score of 5 to 9 on the Mini-Mental State Examination.
